(19) United States
(12) Patent Application Publication
(10) Pub. No.: US 2006/0276795 A1
(43) Pub. Date: Dec. 7, 2006

(54) Scapholunate Disassociation Repair System

(76) Inventors: Jorge L. Orbay, Miami, FL (US); Jose Antonio Nunez, Miami, FL (US); Javier E. Castaneda, Miami, FL (US)

Correspondence Address: GORDON & JACOBSON, P.C., 60 LONG RIDGE ROAD, SUITE 407, STAMFORD, CT, 06902, US

(21) Appl. No.: 11/420,921

(22) Filed: May 30, 2006

Related U.S. Application Data
(60) Provisional application No. 60/686,854, filed on Jun. 2, 2005.

(51) Int. Cl. A61B 17/80; A61F 2/08; A61F 2/42

(52) U.S. Cl. 606/71; 623/13.14; 623/21.11

(57) ABSTRACT

One or more small plates is provided for the fixation of the scapholunate interval between scaphoid and lunate. In one embodiment, a plate is fixed to the bones with fixed angle fasteners to allow complete healing and remodeling of the scapholunate ligament. In another embodiment, the system includes two small plates for the scaphoid and lunate joined by a central element. In yet another embodiment, the system includes two bone anchors for the lunate and scaphoid, also joined by a central element. The central element is flexible, and/or the plates or anchors are movably joined to the central element. The central element in each of these embodiments acts like an artificial ligament and permits more normal motion of the wrist bones while maintaining the scapholunate interval at its correct anatomical dimension.

### CROSS-REFERENCE TO RELATED APPLICATION

This application claims the benefit of U.S. Provisional Application 60/686,854, filed Jun. 2, 2005, which is hereby incorporated by reference herein in its entirety.

### BACKGROUND OF THE INVENTION

1. Field of the Invention

This invention relates broadly to surgery. More particularly, this invention relates to orthopedic implants and methods for the repair of the scapholunate ligament.

2. State of the Art

The rupture of the scapholunate ligament is a well-known clinical problem and can result from trauma or attrition. The scapholunate ligament ties together the scaphoid and lunate bones and is a keystone for stability of the carpal bones. Referring to Prior Art FIG. 1, malfunction of the scapholunate ligament often results in severe carpal dissociation (with separation of the scaphoid 10 and lunate 12) and derangement of the carpal kinematics.

Historically, attempts to surgically repair or reconstruct the ruptured scapholunate ligament have been unsuccessful. The reason for this is not well understood. One hypothesis is that (i) the ligament carries very high forces during normal use; (ii) the ligament is very short (only 2-3 mm in length) and therefore any small deformation is a very high percentage of the entire length of the whole ligament resulting in a large amount of strain; and (iii) there has been no adequate form of immobilization for this small joint.

Referring to Prior Art FIG. 2, conventional treatment for scapholunate disassociation repair utilizes K-wires 14 inserted laterally through the scaphoid 10 and into the lunate 12 to immobilize the joint and permit ligament healing. However, the K-wires 14 tend to move, do not provide sufficient immobilization at the required location, and need to be removed prior to completion of the prolonged healing process.

Referring to Prior Art FIG. 3, screws 16 have also been used for temporary scapholunate fixation. Like K-wires, the screw is inserted in the medial-lateral plane, through the scaphoid 10 and lunate 12 to provide immobilization during ligament healing. This technique also has a high failure rate due to screw loosening and backing out.

There are many soft tissue techniques for repair of the scapholunate ligament, each making an attempt to suture the ligament or reconstruct it by bringing in new ligament tissue. They all need a form of temporary fixation of the scapholunate joint for success. Little progress has been made in providing the surgeon with a better form of temporary fixation.

### SUMMARY OF THE INVENTION

It is therefore an object of the invention to provide system and method for scapholunate ligament repair.

It is another object of the invention to provide a system and method for scapholunate ligament repair that will provide more satisfactory fixation than previously available.

In accord with these objects, which will be discussed in detail below, according to one embodiment of the invention, a small contoured fixed angle plate is provided for the temporary fixation of the scapholunate interval between scaphoid and lunate carpal bones. The plate is fixed to the bones with the fixed angle threaded pegs (locking screws). The plate and screw system is implanted for a semi-permanent amount of time allowing complete healing and remodeling of the scapholunate ligament. The system creates a temporary or reversible arthrodesis which allows almost full function of the hand without cast support. The system can optionally be removed post-healing to allow full mobility of the wrist.

According to another embodiment, the system includes two small plates, one for the scaphoid and the other for the lunate. The two plates are coupled by a metal or polymeric flexible element. According to yet another embodiment, the system includes two bone anchors, one on the scaphoid and the other on the lunate. These anchors are joined by a flexible element. The flexible element in each of these embodiments acts like an artificial ligament and permits more normal motion of the wrist bones while maintaining the scapholunate interval at its correct anatomical dimension.

Additional objects and advantages of the invention will become apparent to those skilled in the art upon reference to the detailed description taken in conjunction with the provided figures.

### BRIEF DESCRIPTION OF THE DRAWINGS

PRIOR ART FIG. 1 is an X-ray volar view of a right hand showing a scapholunate dissociation as a result of a rupture of the scapholunate ligament;

PRIOR ART FIG. 2 is a fluoroscopic volar view of a right hand showing the use of K-wires to treat a scapholunate dissociation;

PRIOR ART FIG. 3 is an X-ray volar view of a left hand showing the use of a bone screw to treat a scapholunate dissociation;

FIG. 4 is a top view of a first embodiment of a plate for a scapholunate disassociation repair system;

FIG. 5 is a proximal-distal view of the plate of FIG. 4;

FIG. 6A is a constructed X-ray volar view of a right hand showing a scapholunate disassociation repair system including the plate of FIG. 4 implanted in a wrist;

FIG. 6B is a constructed X-ray medial view of a right hand showing a scapholunate disassociation repair system including the plate of FIG. 4 implanted in a wrist;

FIG. 7 is a top view of a second embodiment of a plate of a scapholunate disassociation repair system;

FIG. 8 is a proximal-distal view of the plate of FIG. 7;

FIG. 9 is a constructed X-ray volar view showing an implanted scapholunate disassociation repair system according to a third embodiment of the invention;

FIG. 10 is a dorsal view of a fourth embodiment of a scapholunate disassociation repair system;

FIG. 11 is a dorsal view of a fifth embodiment of a scapholunate disassociation repair system;

FIG. 12 is a constructed X-ray volar view showing an implanted scapholunate disassociation repair system according to a sixth embodiment of the invention; and

FIG. 13 is a schematic view of an implanted scapholunate disassociation repair system according to a seventh embodiment of the invention.

### DETAILED DESCRIPTION

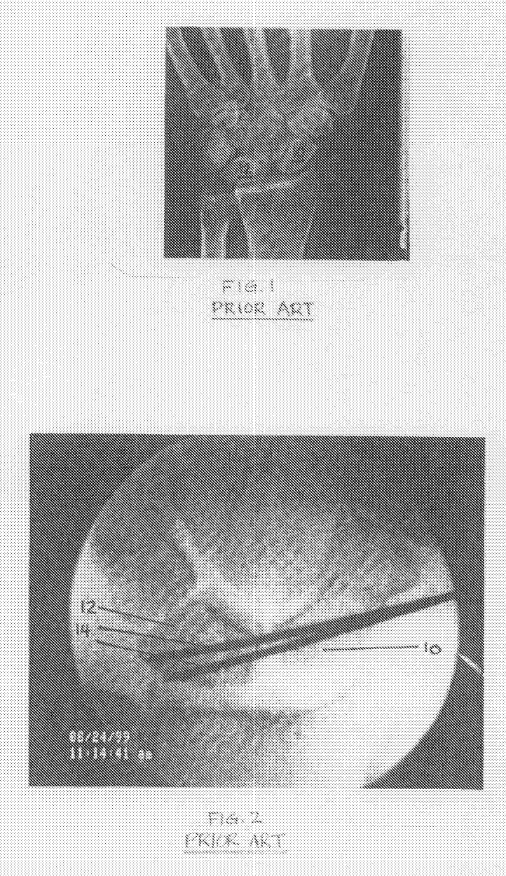

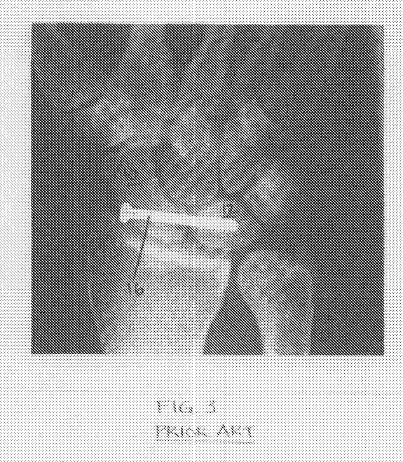

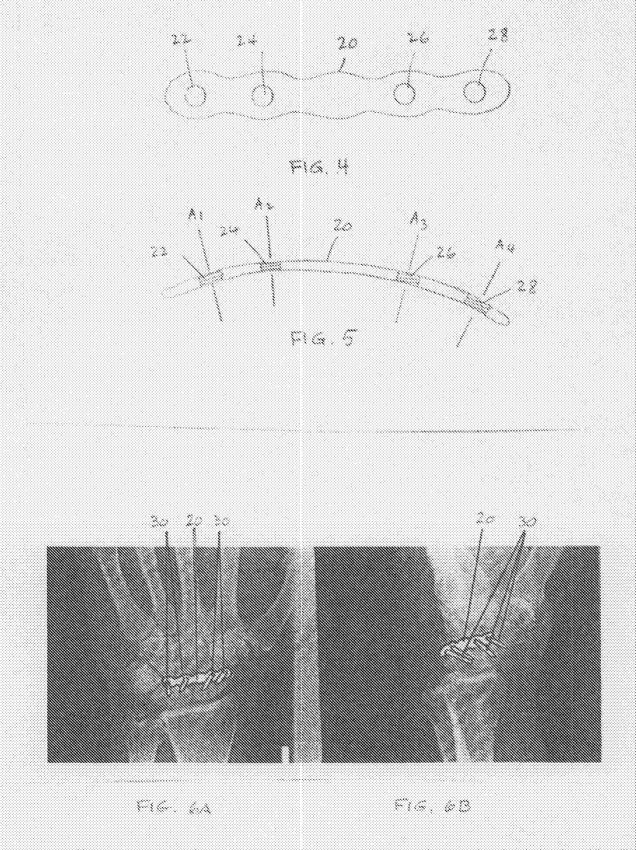

Turning now to FIGS. 4 and 5, according to a first embodiment of the invention, a small contoured fixed angle plate 20 is provided for the temporary fixation of the scapholunate interval between the scaphoid and lunate carpal bones. The plate 20 is preferably curved for placement on the dorsal anatomy of the scaphoid and lunate bones. That is, it is curved both concavely at the bone contacting surface and side-to-side, as described further below. The plate 20 includes a plurality of holes 22, 24, 26, 28 for receiving fasteners 30 (FIGS. 6A and 6B) at a fixed angle relative to the plate. To achieve fixed angle fixation holes 22-28 are preferably threaded, and the heads of the fasteners are threaded to engage the holes. Other means to fix the fasteners relative to the plate may also be used. The shafts of the fasteners are also preferably threaded for rigid engagement with the carpal bones. Holes 22, 24 receive fasteners for insertion into the scaphoid, while holes 26, 28 receive fasteners for insertion into the lunate. Axes A₁, A₂ through holes 22, 24 are preferably obliquely oriented relative to axes A₃, A₄, so that the fasteners conform to the scaphoid and lunate anatomy. In addition, axes A₁ and A₂ may be oblique relative to each other, and axes A₃ and A₄ may also be oblique relative to each other. The plate maintains an optimum dorsal scapholunate interval during healing; i.e., preferably approximately 4.2 mm, in accord with the average dorsal scapholunate interval, but the plate can be provided in sizes to accommodate other common scapholunate intervals, e.g., in the range from 2.3 to 6.3 mm.

The plate 20 and fasteners 30 are implanted on a dorsal side of the scaphoid and lunate bones preferably for at least a semi-permanent amount of time (preferably at least nine months), allowing complete healing and remodeling of the scapholunate ligament. The system creates a temporary or reversible arthrodesis which allows almost full function of the hand without cast support. The system can optionally be removed post-healing to allow full mobility of the wrist.

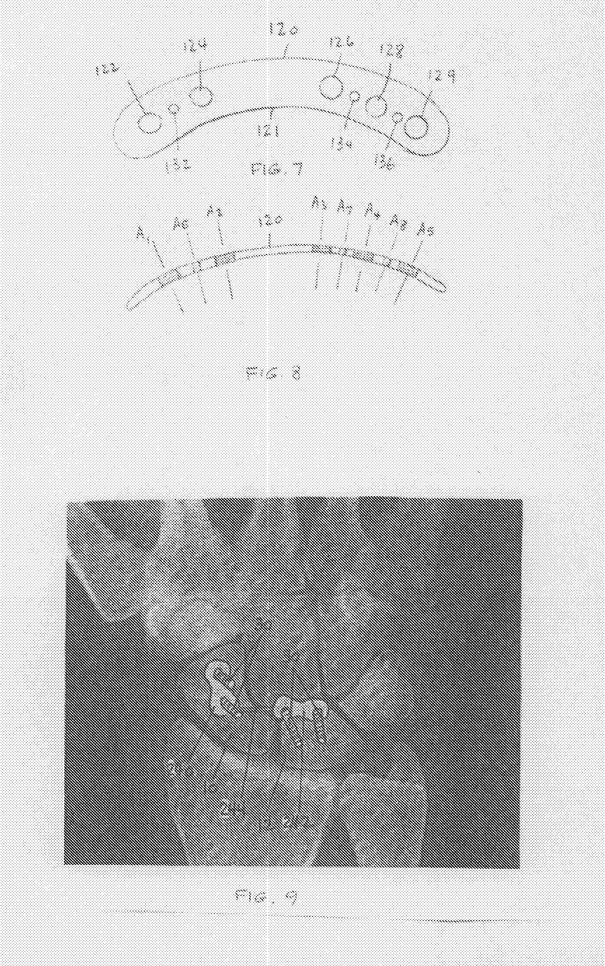

Turning now to FIGS. 7 and 8, a second embodiment of a plate 120 (substantially similar to plate 20) for a scapholunate disassociation repair system is shown. The plate 120 is curved side-to-side (see at distal side 121), preferably along a radius of 30-35 mm, to better fit the scaphoid and lunate bones. Two fastener holes 122, 124 are provided for the lunate, while three fastener holes 126, 128, 129 are provided for the relatively larger scaphoid. Smaller non-threaded alignment holes 132, 134, 136 sized to receive K-wires in a fixed angle orientation are provided between the fastener holes. K-wires can be inserted through the alignment holes 132, 134, 136 to provide temporary fixation and fluoroscopic visualization of the plate and intended approximate trajectories of the fasteners. As the axes A₆, A₇ and As through the respective alignment holes are centered between the axes of the surrounding fastener holes A₁, A₂, A₃, A₄ and A₅, K-wires inserted therethrough provide a good indication of whether the fasteners will be properly inserted prior to implantation. If visualization reveals that intended implantation requires modification, the K-wires can easily be removed and the plate repositioned until the location is appropriate. Removal of the K-wires creates far less tissue disruption than removal of an implanted fastener.

Referring to FIG. 9, according to a third embodiment, the system for scapholunate disassociation repair includes two small plates, one plate 240 for the scaphoid and the other plate 242 for the lunate. Each plate 240, 242 is attached to its respective bone using fasteners 30 as described above. The two plates 240, 242 are attached by a flexible element 244. The flexible element 244 may be, by way of example, a metal braided cable, metal monofilament wire, polymeric filament, polymeric band, polymeric braid, or a combination thereof which is fixedly coupled to each plate via welding, brazing, soldering, bonding, crimping, tying, a set screw, or any other suitable means. Other suitable flexible elements may similarly be used. The flexible element 244 functions like an artificial ligament to permit more normal motion of the wrist bones while maintaining the scapholunate interval at its correct anatomical dimension. The scapholunate joint does have some physiologic motion which is quite complex and has a center of rotation outside of the body of either the scaphoid and lunate. This motion includes translation and rotation and can only be allowed by a flexible element 244 that has the insertion points of the scapholunate ligament.

The system of FIG. 9 accurately reproduces the insertion sites of the scapholunate ligament and allows for very normal kinematics while the healing process occurs. Like all the other embodiments, the elements of the system can be permanent or removed after ligament healing, usually nine months post-operative.

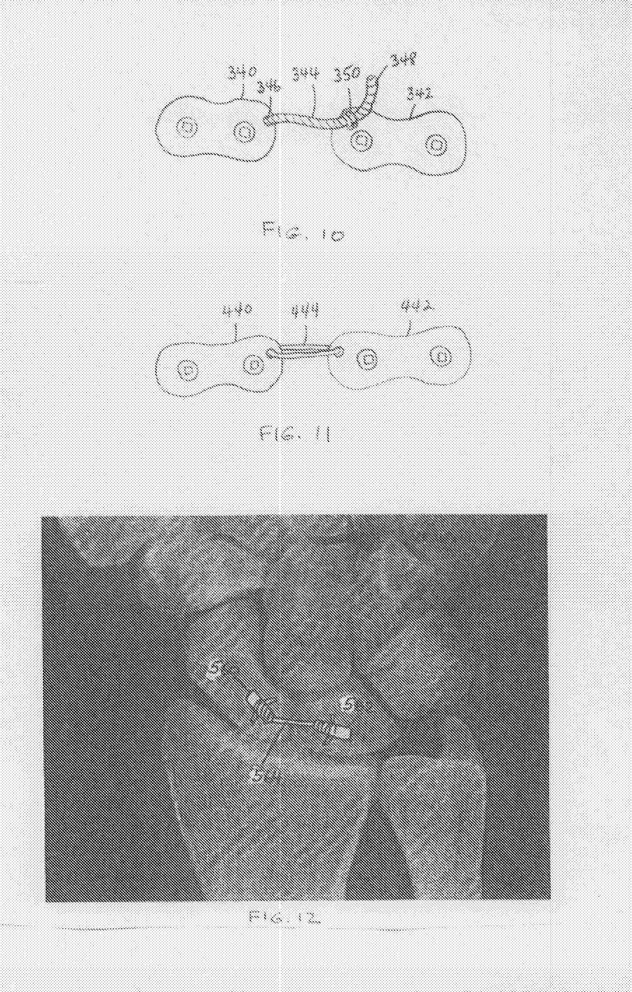

Turning now to FIG. 10, a fourth embodiment of the system is substantially the same as the third embodiment described with respect to FIG. 9. In the fourth embodiment of FIG. 10, one end 346 of the flexible element 344 is fixedly coupled to one plate, e.g., 340, while the other end (free end) 348 may be moved relative to the second plate, e.g., 342 during implantation. Then, once both the plates are implanted on the scaphoid and lunate, and the scaphoid and lunate are anatomically positioned, the free end 348 is pulled and the second plate 342 is locked relative to the flexible element to maintain the scapholunate interval. The flexible element and plate are locked relative to each other using, e.g., deformation crimping of a band 350 through which the flexible element 344 extends or rotation of a set screw which compresses the flexible element. In this manner, the procedure is facilitated by providing additional slack between the plates during plate implantation, but the ability to correct the anatomical derangement is not compromised.

In an alternate embodiment, both plates may be movable relative to the flexible element and then lockable in position. In yet another alternate embodiment, the plates may be discretely coupled to the scaphoid and lunate (without any flexible element extending therebetween), and the flexible element may thereafter be introduced and coupled to the plates.

Turning now to FIG. 11, in a fifth embodiment, similar to the third embodiment, the plates 440, 442 may be movably joined to a relatively rigid link 444 (or other substantially rigid member) which spaces the plates at the appropriate scapholunate interval and allows the plates to move in the desired degrees of freedom.

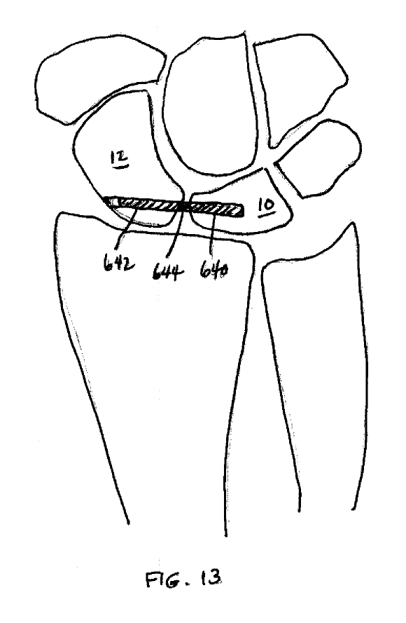

Turning now to FIG. 12, according to a sixth embodiment, the system for scapholunate disassociation repair is plateless, including two bone anchors, one anchor 560 on the scaphoid and the other anchor 562 on the lunate, joined by a flexible element 564. The fifth embodiment functions similarly to the third embodiment and has the same advantages. Similarly, the anchors and flexible element may be configured such that at least one anchor is movable relative to an end of the flexible element (to facilitate implantation), moved thereto upon alignment of the anatomy, and then locked relative thereto to maintain the scapholunate interval. It is appreciated that the plateless embodiment lends itself to an arthroscopic approach to scapholunate disassociation repair. Alternatively, the bone anchors may be movably joined to a non-flexible central element, as discussed above with respect to the fifth embodiment.

Referring now to FIG. 13, another system for scapholunate disassociation repair is shown. The system is adapted for percutaneous repair, including first and second screw portions 640, 642, and a flexible member 644 therebetween. The first and second screw portions 640, 642 are initially coupled together. The first screw portion 640 is percutaneously introduced through the lunate bone and into the scaphoid bone 10, and the second screw portion 642 is percutaneously introduced into the lunate bone 12. The first and second screw portions are then decoupled. The flexible member 644 extends between the screw portions 640, 642 and thus the two bones 10, 12 to hold the scaphoid and lunate in relation at the appropriate scapholunate interval, while permitting the desired degrees of freedom between the bones.

There have been described and illustrated herein several embodiments of a scapholunate disassociation repair system and methods for repairing scapholunate disassociations. While particular embodiments of the invention have been described, it is not intended that the invention be limited thereto, as it is intended that the invention be as broad in scope as the art will allow and that the specification be read likewise. While fixed angle threaded peg fasteners have been described for use with the plate, it will be appreciated that other fasteners including smooth shaft pegs, fixed angle fasteners which do not require a threaded head engaging a threaded hole, and even non-fixed angle fasteners may be used with the plates described herein, such plates being modified as necessary for use with such fasteners. It will therefore be appreciated by those skilled in the art that yet other modifications could be made to the provided invention without deviating from its spirit and scope as claimed.

What is claimed is:

1. A scapholunate disassociation repair system, comprising:
a) a first plate sized for placement on a scaphoid bone, said first plate including at least one fixed angle hole therein;
b) a second plate sized for placement on a lunate bone, said second plate including at least one fixed angle hole therein; and
c) an element coupling said first and second plates together so that said first and second plates are spaced apart a distance approximating a dorsal scapholunate interval.

2. A system according to claim 1, further comprising:
fixed angle fasteners for insertion through said fixed angle holes in said first and second plates for coupling said first and second plates to said scaphoid bone and said lunate bone respectively.

3. A system according to claim 1, wherein:
said element is rigid.

4. A system according to claim 1, wherein:
said element is flexible.

5. A system according to claim 4, wherein:
said element is adjustable in length.

6. A system according to claim 1, wherein:
said first plate includes two fixed angle holes each defining a central axis, said central axes of said two holes being obliquely angled relative to each other.

7. A system according to claim 6, wherein:
said second plate includes two fixed angle holes each defining a central axis, said central axes of said two holes of said second plate being obliquely angled relative to each other.

8. A scapholunate disassociation repair system, comprising:
a) a first plate sized for placement on a scaphoid bone, said first plate including at least one fixed angle hole therein;
b) a second plate sized for placement on a lunate bone, said second plate including at least one fixed angle hole therein; and
c) an element coupling said first and second plates together so that said first and second plates when coupled to the scaphoid and lunate bones cause said scaphoid and lunate bones to be spaced apart a dorsal scapholunate interval.

9. A system according to claim 8, further comprising:
fixed angle fasteners for insertion through said fixed angle holes in said first and second plates for coupling said first and second plates to said scaphoid bone and said lunate bone respectively.

10. A system according to claim 8, wherein:
said element is rigid.

11. A system according to claim 8, wherein:
said element is flexible.

12. A system according to claim 11, wherein:
said element is adjustable in length.

13. A scapholunate disassociation repair system, comprising:
a) a first bone fastener;
b) a second bone fastener; and
c) an element coupling said first and second bone fasteners together, said coupling element having a length such that dorsal surfaces of scaphoid and lunate bones to which said first and second bone fasteners are to be coupled are spaced apart a dorsal scapholunate interval.

14. A system according to claim 13, wherein:
said element is flexible.

15. A system according to claim 13, wherein:
said element is a plate having a bone contacting surface with a concave curvature in a direction of its length.

16. A system according to claim 13, wherein:
said plate is curved side-to-side.

17. A system according to claim 11, wherein:
said curve has a radius of approximately 30-35 mm.

18. A method of scapholunate repair, comprising:
coupling the scaphoid and lunate bone together in a manner which maintains the dorsal scapholunate interval between the scaphoid and lunate bones while permitting movement of the scapholunate joint.

19. A method according to claim 18, wherein:
said coupling includes attaching discrete plates to said scaphoid and lunate bones, said plates being coupled together with a flexible element.

20. A method according to claim 19, further comprising:
adjusting a length of said flexible element.

21. A method of scapholunate repair, comprising:
coupling the scaphoid and lunate bones together in a manner which maintains the dorsal scapholunate interval between the scaphoid and lunate bones, said coupling using at least one fixed angle fastener extending into each of the scaphoid and lunate bones.

22. A method according to claim 21, wherein:
said coupling includes positioning a plate across the scaphoid and lunate bones and inserting fixed angle fasteners through said plate and into the scaphoid and lunate bones.

23. A method according to claim 21, wherein:
said coupling includes positioning a scaphoid plate on the scaphoid bone and a lunate plate on the lunate bone, said scaphoid and lunate plates being coupled by a flexible element, and inserting fixed angle fasteners through said respective scaphoid and lunate plates and into the scaphoid and lunate bones.
